(19) United States
(12) Patent Application Publication
(10) Pub. No.: US 2025/0325403 A1
(43) Pub. Date: Oct. 23, 2025

(54) PHACO DRIVER SYSTEM, A METHOD AND A COMPUTER PROGRAM PRODUCT

(71) Applicant: D.O.R.C. Dutch Ophthalmic Research Center (International) B.V., Zuidland (NL)

(72) Inventor: Kees Bijkerk, Dordrecht (NL)

(21) Appl. No.: 19/255,138

(22) Filed: Jun. 30, 2025

Related U.S. Application Data
(63) Continuation of application No. PCT/EP2023/087975, filed on Dec. 29, 2023.

(30) Foreign Application Priority Data
Dec. 30, 2022 (NL) 2033887

(51) Int. Cl. A61F 9/007; A61B 17/00

(52) U.S. Cl. CPC A61F 9/00745; A61B 2017/0015

(57) ABSTRACT

A phaco driver system is for controlling a dual frequency operation of an ophthalmic surgical phacoemulsification device. The phaco driver system includes a bridge unit generating a bridge output signal for driving an ultrasonic transducer of the ophthalmic surgical phacoemulsification device. The bridge output signal is a block signal based on a sum of a first input signal having a first frequency and a second input signal having a second frequency. The phaco driver system also includes a control unit setting parameters of the first input signal and parameters of the second input signal such that the bridge output signal includes a first component having the first frequency and a second component having the second frequency, the first and the second component having a selectable output ratio.

### CROSS-REFERENCE TO RELATED APPLICATIONS

This application is a continuation application of international patent application PCT/EP2023/087975, filed Dec. 29, 2023, designating the United States and claiming priority from NL application 2033887, filed Dec. 30, 2022, and the entire content of both applications is incorporated herein by reference.

### TECHNICAL FIELD

The disclosure relates to a phaco driver system.

Phacoemulsification is known as a process for disintegration of the lens of an eye utilizing a ophthalmic surgical phacoemulsification device vibrating at ultrasonic frequencies. Such phacoemulsification device may include an ultrasonic transducer operatively connected to a needle having a cutting tip which is vibrated at ultrasonic frequencies to disintegrate cataractic tissue in the eye.

A phaco driver system controls operation of the ophthalmic surgical phacoemulsification device, typically by generating a transducer voltage for driving an ultrasonic transducer. Thereto, the phaco driver system may include a resonance output circuit to produce the transducer voltage. During operation a surgeon may desire to apply, via the ultrasonic transducer such as a piezo, either a longitudinal motion or a transverse motion of the cutting tip, or a combination of the longitudinal motion and the transverse motion so as to perform different cutting modalities. Even further motion types may be used, for example, a torsional motion of the cutting tip. In known phaco driver systems, multiple frequencies may be selectively generated to drive a specific motion types, or a combination of motion types.

The ophthalmic surgical phacoemulsification device may include multiple piezos, for example, a first piezo for performing a longitudinal movement, and a second piezo for performing a transverse or torsional movement. Alternatively, the ophthalmic surgical phacoemulsification device may include a single piezo contacting two mechanical resonance parts tuned to different frequencies. In the former structure multiple feeding lines are used, each feeding line feeding a respective piezo. In the latter structure a single feeding line may suffice for driving the device in multiple cutting modalities.

In patent publication U.S. Pat. No. 9,572,711 B2 a system for performing an ocular surgical procedure is disclosed including two driver modules arranged in parallel each driver module generating a single frequency signal so as to drive an ophthalmic surgical phacoemulsification device via a single transformer at two superimposed frequencies.

### SUMMARY

It is an object of the present disclosure to provide a phaco driver system for controlling dual frequency operation of an ophthalmic surgical phacoemulsification device. It is further an object of the present disclosure to provide a phaco driver system for controlling dual frequency operation of an ophthalmic surgical phacoemulsification device wherein the number of components of the phaco driver system is reduced enabling a more compact configuration. It is further an object of the present disclosure to provide a phaco driver system in which the power requirement may be minimal, leading to even smaller components and/or reducing necessity of heatsinks and resulting therefore in an even more compact configuration. Thereto, according to the disclosure, a phaco driver system is provided for controlling dual frequency operation of an ophthalmic surgical phacoemulsification device, including a bridge unit for generating a bridge output signal for driving an ultrasonic transducer of the ophthalmic surgical phacoemulsification device, the bridge output signal being a block signal based on a sum of a first input signal and a second input signal, and a control unit for generating the first input signal and the second input signal, wherein the first signal is a harmonic signal having a first frequency and wherein the second signal is a harmonic signal having a second frequency, wherein the control unit is configured to set parameters of the first signal and parameters of the second signal such that the bridge output signal includes a first component having the first frequency and a second component having the second frequency, the first and the second component having a selectable output ratio. The bridge output signal preferably, but not necessarily, is an alternating block signal. The sum of the first input signal and the second input signal preferably, but not necessarily, is a logic sum.

By providing, according to an aspect of the disclosure, a control unit generating a first input signal having a first frequency and a second input signal having a second frequency for a bridge unit that drives the ultrasonic transducer of the ophthalmic surgical phacoemulsification device, and by setting parameters of the first signal and parameters of the second signal such that the bridge output signal includes a first component having the first frequency and a second component having the second frequency, the first and the second component having a selectable output ratio, a spectral behavior of the bridge output signal can be controlled, in particular to select a spectral characteristic of the bridge output signal. As an example, the first frequency component of the bridge output signal may dominate over the second frequency component, or vice versa, or both frequency components may mainly balance with each other. Then, the phaco driver system may, on the one hand, offer dual frequency controlling features while, on the other hand, requires a single bridge unit only in its implementation, thereby reducing its number of components enabling a more compact configuration.

According to an aspect of the disclosure, the selectable output ratio of the respective frequency components in the bridge output signal is controlled by the control unit setting parameters of the first and second input signals, in particular their amplitude ratio, thus exploiting a transfer function, such as a non-linear transfer function, of the bridge unit. In principle, the output ratio of the respective frequency components in the bridge output signal may be selected from a continuous range of ratio values, for example, from circa 0.01 to circa 100. Alternatively, a discrete number of output ratios of the respective frequency components in the bridge output signal may be selected, for example, three output ratios.

Typically, the bridge output signal is block-shaped having a first harmonics with a frequency that mainly coincides with the first frequency, in the first operation mode, or with the second frequency, in the second operation mode, respectively.

The control unit may operate in at least a first and a second operation mode wherein an amplitude ratio of the first and the second input signal is either above a first threshold value or below a second threshold value, respectively, for selecting a output ratio that is larger or smaller than unity, respectively.

Advantageously, the first threshold value may be greater than unity and the second threshold value may be smaller than unity. As an example, the first threshold value may be circa 1.5 while the second threshold value may be circa ⅔. Then, a user of the phaco driver system may select a desired cutting motion type of the phacoemulsification device by selectively modifying or setting the amplitude ratio of the first and second input signals, thereby selecting a desired frequency in the spectrum of the bridge output signal.

Especially, the control unit may be arranged for selectively operating in a third operation mode wherein an amplitude ratio for the first and the second input signals is circa unity, providing a bridge output signal having a frequency spectrum including both the first frequency and the second frequency for driving the phacoemulsification device in both motion types, for example, in longitudinal and transverse cutting tip motion.

In addition, the disclosure relates to a method.

Further, the disclosure relates to a computer program product. A computer program product may include a set of computer executable instructions stored on a data carrier, such as a CD or a DVD. The set of computer executable instructions, which allows a programmable computer to carry out the method as defined above, may also be available for downloading from a remote server, for example via the Internet, for example, as an app.

It should be noted that the technical features described above or below may each on its own be embodied in a system or method, that is, isolated from the context in which it is described, separate from other features, or in combination with only a number of the other features described in the context in which it is disclosed. Each of these features may further be combined with any other feature disclosed, in any combination.

### BRIEF DESCRIPTION OF DRAWINGS

The invention will now be described with reference to the drawings wherein:

FIG. 1 shows a schematic view of a phaco driver system according to the disclosure;

FIG. 2 shows a diagram including signals in the system shown in FIG. 1, in a first operation mode;

FIG. 3 shows a diagram including signals in the system shown in FIG. 1, in a second operation mode;

FIG. 4 shows a diagram including signals in the system shown in FIG. 1, in a third operation mode;

FIG. 5A shows a spectral diagram of the bridge output signal in the first operation mode;

FIG. 5B shows a spectral diagram of the bridge output signal in the second operation mode;

FIG. 5C shows a spectral diagram of the bridge output signal in the system in the third operation mode, and

FIG. 6 shows a flow chart of a method according to the disclosure.

### DETAILED DESCRIPTION

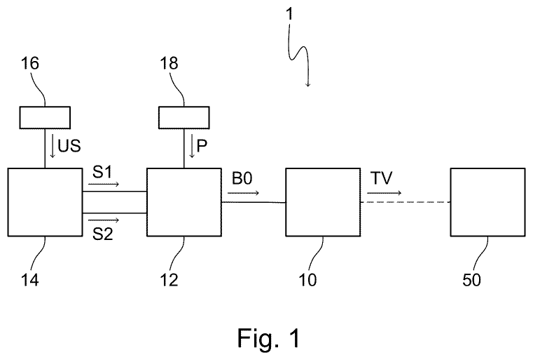

FIG. 1 shows a schematic view a phaco driver system 1 according to the disclosure. The phaco driver system 1 is arranged for controlling dual frequency operation of an ophthalmic surgical phacoemulsification device 50, for example, for performing different types of motion, for example, longitudinal motion, transverse motion or torsional motion, or a combination of motions.

The phaco driver system 1 includes an optional resonance output circuit 10 for generating a transducer voltage TV. The transducer voltage TV may be used for driving an ultrasonic transducer of the ophthalmic surgical phacoemulsification device 50, for example, a phacoemulsification hand piece device shown in FIG. 1.

The phaco driver system 1 also includes a bridge unit 12 for generating a bridge output signal BO. The bridge unit 12 may be implemented using an H-bridge or another bridge unit, typically a unit converting analogue signals to a digital bridge output signal BO. The output signal BO may be fed to the resonance output circuit 10 that may be arranged for filtering out unwanted harmonics of the bridge output signal BO. The bridge output signal BO is based on a sum of a first input signal S1 and a second input signal S2 that are received from a control unit 14 described below.

The phaco driver system 1 further includes a control unit 14. The control unit 14 is arranged for generating the first input signal S1 and the second input signal S2, wherein the first signal S1 is a harmonic signal having a first frequency f1 and wherein the second signal S2 is a harmonic signal having a second frequency f2.

As an example, the first frequency f1 is circa 32 kHz, while the second frequency f2 is circa 44 kHz. It is noted that the first and/or second frequency f1, f2 may have another value, for example, more than 44 kHz or less than 32 kHz.

Generally, the control unit 14 is configured to set parameters of the first input signal S1 and parameters of the second input signal S2 such that the bridge output signal BO includes a first component having the first frequency f1 and a second component having the second frequency f2, the first and the second component having a selectable output ratio.

The control unit 14 of the described embodiment is further arranged for selectively operating in at least a first operation mode wherein an amplitude ratio R of the first and second input signals S1, S2 is above a first threshold value thr1, or a second operation mode wherein an amplitude ratio R of the first and second input signals S1, S2 is below a second threshold value thr2. The amplitude ratio R may be defined as the maximum amplitude A1 of the first signal S1 divided by the maximum amplitude A2 of the second signal S2, in short R=A1/A2.

In the embodiment shown in FIG. 1, the phaco driver system 1 also includes a user interface 16 enabling a user of the system 1 to select an operation mode of the control unit 14 by sending a user signal US to the control unit 14. Optionally, the control unit 14 may be connected to a CPU or programmable computer, for example, for receiving control signals.

Similarly, the embodiment shown in FIG. 1, the phaco driver system 1 has a power supply unit 18 for generating a power supply P to the bridge unit 12.

Preferably, the bridge output signal BO is a composed block signal, for example, taking values in the set {−V, 0, +V} with V a voltage amplitude related to the power supply P.

The resonance output circuit 10, the bridge unit 12, the control unit 14 and/or the power supply unit 18 of the phaco driver system 1 may be integrated in a single hardware component. For example, the control unit 14 may be realized in an FPGA which is housed within the same chip as a CPU.

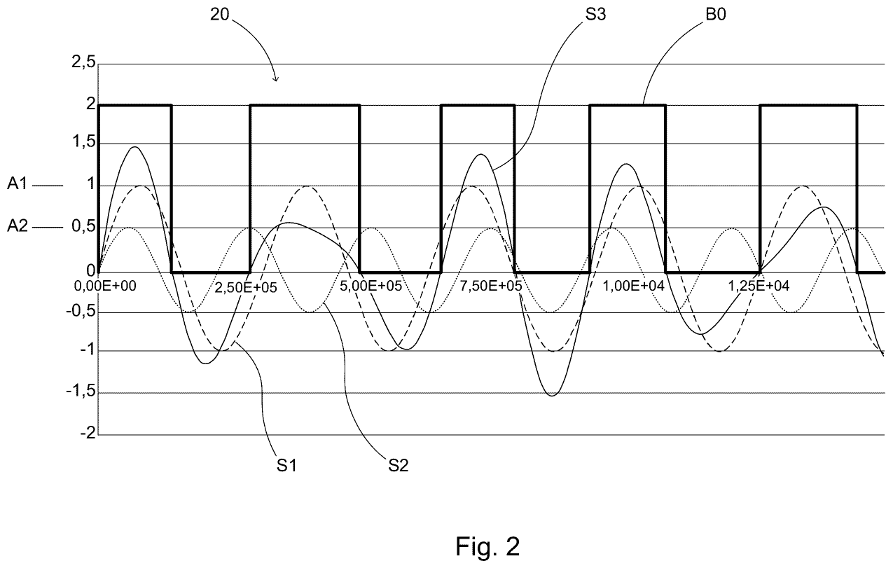

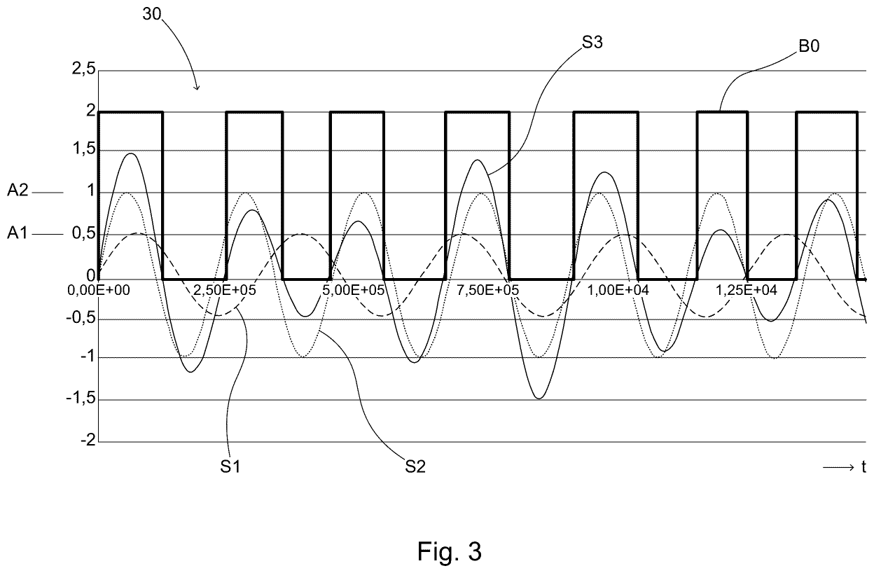

FIG. 2 shows a diagram including signals in the system 1 shown in FIG. 1, in a first operation mode, while FIG. 3 shows a diagram including signals in the system 1 shown in FIG. 1, in a second operation mode.

In FIG. 2, the diagram 20 shows the first signal S1 having an amplitude A1 and a frequency f1, as well as the second signal S2 having an amplitude A2 and a frequency f2, both as a function of time t. The diagram 20 further shows a sum signal S3 being the result of adding the first signal S1 to the second signal S2. The diagram 20 also shows the bridge output signal BO being a block shaped signal.

The bridge output signal BO is based on the sum of the first input signal S1 and a second input signal S2, that is, based on the sum signal S3. Upon the zero-crossings of the sum signal S3 the bridge output signal BO changes.

In the first operation mode, the amplitude ratio R of the first and second input signals S1, S2 is above a first threshold value thr1. Here, the amplitude ratio R being the maximum amplitude A1 of the first signal S1 divided by the maximum amplitude A2 of the second signal S2, such that R is approximately 2.

The first threshold value thr1 may be set to be typically equal to or greater than unity, for example, 1.5. In the first operation mode described referring to FIG. 2 the amplitude ratio R is greater than the first threshold value thr1.

It appears that, in the first operation mode, the bridge output signal BO has a first harmonics that mainly coincides with the first frequency f1.

Upon feeding the first and the second signals S1, S2 to the bridge unit 12, a comparison of the input signal amplitudes A1, A2 drives the bridge unit 12 to obtain an output signal based on the sum of the two input signals S1, S2. Here, a contribution of the individual input signals S1, S2 in the bridge output signal BO is based on the amplitude ratio R. In the shown embodiment, the bridge output signal BO may be directly digitally generated. Advantageously, a minimum of hardware components may be used.

The bridge output signal BO shown in the diagram 20 of FIG. 2 is block-shaped having a first harmonics that is mainly similar to the first frequency f1, that is, the frequency of the first signal S1.

In FIG. 3, showing a diagram 30 with signals, in a second operation mode, the amplitude ratio R being the maximum amplitude A1 of the first signal S1 divided by the maximum amplitude A2 of the second signal S2, is approximately ½.

The second threshold value thr2 may be set to be typically equal to or smaller than unity, for example, ⅔. In the second operation mode described referring to FIG. 3 the amplitude ratio R is smaller than the second threshold value thr2.

It appears that, in the second operation mode, the bridge output signal BO has a first harmonics that mainly coincides with the second frequency f2, that is, the frequency of the second signal S2.

In a specific example, the second threshold value thr2 may be set to be the inverse of the first threshold value thr1.

As shown, the first harmonics of the bridge output signal BO may be controlled by setting an amplitude ratio R of the first and second input signals S1, S2.

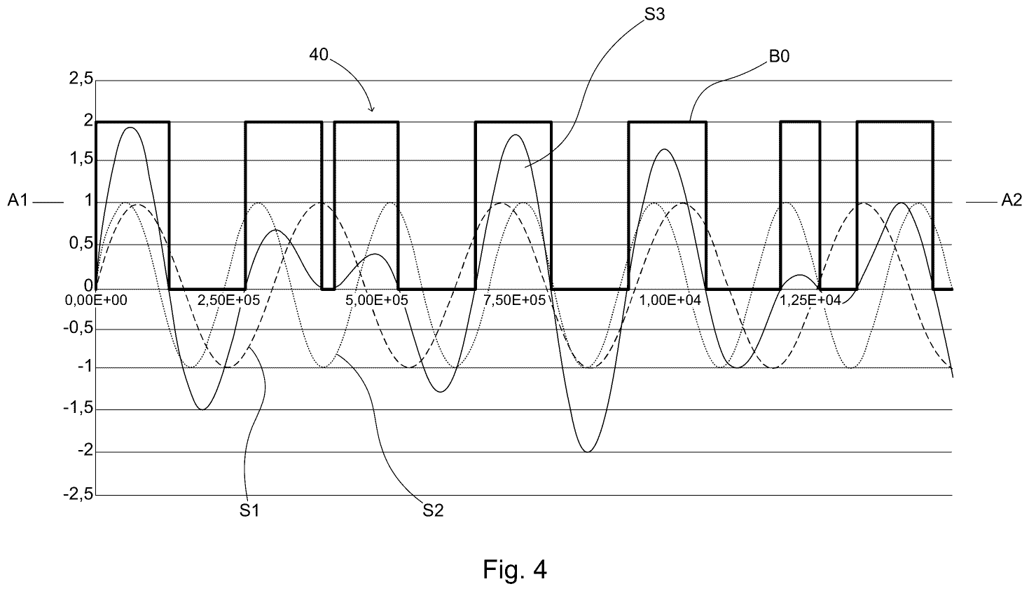

The control unit 14 may be arranged for selectively operating also in a third operation mode wherein the amplitude ratio R of the first and the second signals is circa unity, as described below referring to FIG. 4.

FIG. 4 shows a diagram 40 including signals in the system shown in FIG. 1, in a third operation mode. Here, the maximum amplitude A1 of the first signal S1 is mainly the same as the maximum amplitude A2 of the second signal S2. Then, the amplitude ratio R is circa unity, between the first threshold value thr1 and the second threshold value thr2.

In the third operation mode, the bridge output signal BO has a frequency spectrum including both the first frequency f1 and the second frequency f2.

It is noted that the bridge output signal BO is block shaped having relatively steep rising and falling edges, and having relatively sharp transition sections with upper and lower levels of the blocks. In practice, the rising and falling edges may be less steep, and transition sections with the upper and lower levels of the blocks may be smoothened or rounded.

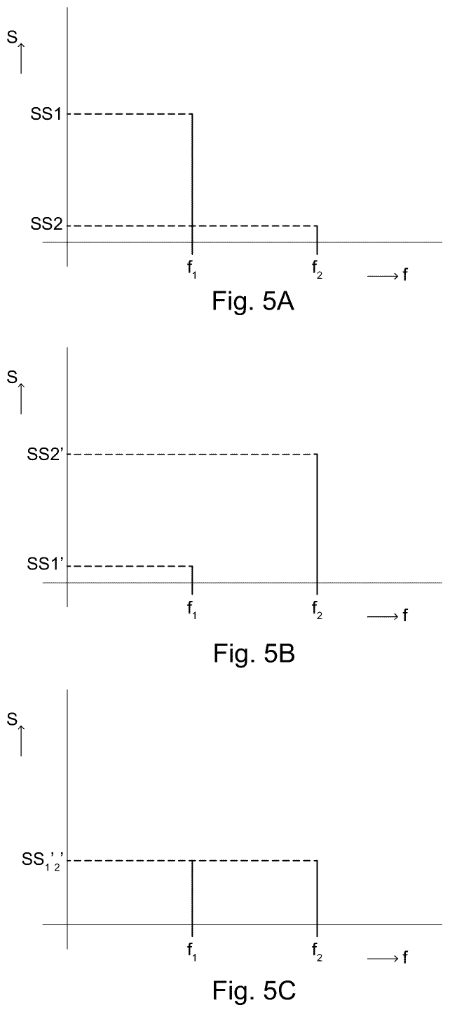

FIGS. 5A to 5C show spectral diagrams of the bridge output signal BO in the first, second and third operation mode, respectively. The diagrams shows the spectral amplitude or power S of individual spectral components of the respective signals as a function of frequency f.

In FIG. 5A, corresponding to the first operation mode, the bridge output signal BO has a first component at the first frequency f1 and a second component at the second frequency f2. The first component has a first spectral amplitude SS1 while the second component has a second spectral amplitude SS2. The first spectral amplitude SS1 is larger than the second spectral amplitude SS2. Here, the first and the second component have a selectable output ratio SS1/SS2 that, in the first operation mode, is larger than unity, for example, circa 3, circa 5, circa 10, circa 50 or more, for example, circa 100. Then, the contribution of the first input signal S1 in the bridge output signal BO is dominant over the contribution of the second input signal S2, due to the parameters of the first and second signals S1, S2, in particular the amplitude ratio R thereof as set by the control unit 14 described above.

In FIG. 5B, corresponding to the second operation mode, the first frequency component of the bridge output signal BO has a first spectral amplitude SS1′ that is smaller than the second spectral amplitude SS2′ of the second frequency component of the bridge output signal BO. Here, the first and the second component have a selectable output ratio SS1/SS2 that, in the second operation mode, is smaller than unity, for example, circa ⅓, circa ⅕, circa 0.1, circa 1/50 or less, for example, circa 0.01. Then, the contribution of the second input signal S2 in the bridge output signal BO is dominant over the contribution of the first input signal S1, due to the parameters of the first and second signals S1, S2, in particular the amplitude ratio R thereof as set by the control unit 14 described above.

In FIG. 5C, corresponding to the third operation mode, the first frequency component of the bridge output signal BO has a first spectral amplitude SS1″ that is mainly equal to the second spectral amplitude SS2″ of the second frequency component of the bridge output signal BO. Here, the first and the second component have a selectable output ratio SS1/SS2 that, in the third operation mode, is circa unity. Then, the contribution of the first input signal S1 and the second input signal S2 in the bridge output signal BO is mainly equal or mainly balance, due to the parameters of the first and second signals S1, S2, in particular the amplitude ratio R thereof as set by the control unit 14 described above.

In principle, the output ratio of the first frequency component and the second frequency component in the bridge output signal BO can be selected by the control unit 14, based on a transfer function, generally a non-linear transfer function, of the bridge unit 12, by setting parameters of the first input signal S1 and parameters of the second input signal S2, in particular by setting the amplitude ratio R of the first and second signals S1, S2. The selectable output ratio SS1/SS2 may generally range from circa 0.01 to circa 100, for example, to the values described referring to FIGS. 5A to 5C, or to values therebetween.

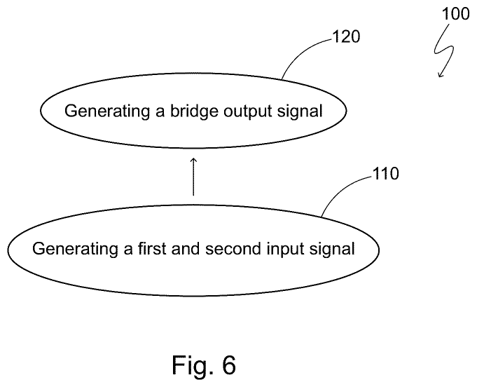

FIG. 6 shows a flow chart of a method according to the disclosure. The method is used for controlling operation of an ophthalmic surgical phacoemulsification device. The method may for example be implemented using the above-described phaco driver system 1. The method 100 includes a step 120 of generating, using a bridge unit, a bridge output signal for driving an ultrasonic transducer of the ophthalmic surgical phacoemulsification device, the bridge output signal being based on a sum of a first input signal and a second input signal, and a step 110 of generating, using a control unit, the first input signal and the second input signal, wherein the first signal is a harmonic signal having a first frequency and wherein the second signal is a harmonic signal having a second frequency, wherein the control unit sets parameters of the first signal and parameters of the second signal such that the bridge output signal includes a first component having the first frequency and a second component having the second frequency, the first and the second component having a selectable output ratio.

The step of generating the first and the second input signals S1, S2 can be performed using dedicated hardware structures, such as FPGA and/or ASIC components. Otherwise, the method can at least partially be performed using a computer program product including instructions for causing a processor of a computer system to perform the above-described steps. The step can in principle be performed on a single processor. However it is noted that at least a substep can be performed on a separate processor, for example, a substep of generating the first input signal S2.

The invention is not restricted to the embodiments described herein. It will be understood that many variants are possible.

The phaco driver system 1 can be implemented without the optional resonance output circuit 10. Then, the bridge unit 12 generates a bridge output signal BO for directly driving the ultrasonic transducer of the ophthalmic surgical phacoemulsification device 50.

It is understood that the foregoing description is that of the preferred embodiments of the invention and that various changes and modifications may be made thereto without departing from the spirit and scope of the invention as defined in the appended claims.

1. A phaco driver system for controlling a dual frequency operation of an ophthalmic surgical phacoemulsification device, the phaco driver system comprising:
a bridge unit configured to generate a bridge output signal for driving an ultrasonic transducer of the ophthalmic surgical phacoemulsification device, the bridge output signal being a block signal based on a sum of a first input signal and a second input signal;
a control unit configured to generate the first input signal and the second input signal, wherein the first signal is a harmonic signal having a first frequency and the second signal is a harmonic signal having a second frequency; and,
said control unit being configured to set parameters of the first input signal and parameters of the second input signal such that the bridge output signal includes a first component having the first frequency and a second component having the second frequency, the first component and the second component having a selectable output ratio.

2. The phaco driver system of claim 1, wherein the control unit is further configured for selectively operating in at least a first operation mode wherein the parameters of the first signal and the parameters of the second signal are set such that an amplitude ratio of the first and second input signals is above a first threshold value, or a second operation mode wherein the parameters of the first signal and the parameters of the second signal are set such that the amplitude ratio of the first and second input signals is below a second threshold value.

3. The phaco driver system of claim 1, wherein the selectable output ratio is selectively configurable in a range between 0.01 and 100.

4. The phaco driver system of claim 1 further comprising a power supply unit for generating a power supply to the bridge unit.

5. The phaco driver system of claim 1, wherein the first threshold value is greater than unity; and, the second threshold value is smaller than unity.

6. The phaco driver system of claim 1, wherein the control unit is configured to selectively operate in a third operation mode wherein an amplitude ratio of the first input signal and the second input signal is circa unity.

7. The phaco driver system of claim 1 further comprising a user interface for selecting an operation mode of the control unit.

8. The phaco driver system of claim 1, wherein a resonance output circuit, the bridge unit and the control unit and/or a power supply unit are integrated in a single hardware component.

9. The phaco driver system of claim 1 further comprising a resonance output circuit configured to be fed by the bridge output signal and to generate a transducer voltage for driving the ultrasonic transducer of the ophthalmic surgical phacoemulsification device.

10. A method of controlling a dual frequency operation of an ophthalmic surgical phacoemulsification device, the method comprising:
generating, via a bridge unit, a bridge output signal for driving an ultrasonic transducer of the ophthalmic surgical phacoemulsification device, the bridge output signal being a block signal based on a sum of a first input signal and a second input signal;
generating, via a control unit, the first input signal and the second input signal, wherein the first signal is a harmonic signal having a first frequency and wherein the second signal is a harmonic signal having a second frequency; and,
wherein the control unit sets parameters of the first signal and parameters of the second signal such that the bridge output signal includes a first component having the first frequency and a second component having the second frequency, the first and the second component having a selectable output ratio.

11. The method according to claim 10 further comprising generating a power supply to the bridge unit.

12. The method of claim 10, wherein the control unit selectively operates in a third operation mode; and, an amplitude ratio of the first input signal and the second input signal is unity.

13. The method of claim 10, wherein, in the first operation mode, the bridge output signal has first harmonics that coincide with the first frequency; and, in the second operation mode, the bridge output signal has second harmonics that coincide with the second frequency.

14. The method of claim 12, wherein, in the third operation mode, the bridge output signal has a frequency spectrum including both the first frequency and the second frequency.

15. A computer program product for controlling a dual frequency operation of an ophthalmic surgical phacoemulsification device, the computer program product comprising computer readable code stored on a non-transitory computer readable medium, the computer readable code being configured to cause a processor to generate, using a control unit, a first input signal and a second input signal, wherein the first signal is a harmonic signal having a first frequency and wherein the second signal is a harmonic signal having a second frequency, for generating, via a bridge unit, a bridge output signal being a block signal based on a sum of the first input signal and the second input signal, for driving an ultrasonic transducer of the ophthalmic surgical phacoemulsification device,
wherein the control unit is configured to set parameters of the first signal and parameters of the second signal such that the bridge output signal includes a first component having the first frequency and a second component having the second frequency, the first and the second component having a selectable output ratio.
